(19) United States
(12) Patent Application Publication
(10) Pub. No.: US 2008/0317677 A1
(43) Pub. Date: Dec. 25, 2008

(54) Laser Marked Dosage Forms

(76) Inventors: Christopher E. Szymczak, Marlton, NJ (US); Irvin Matas Lash, Pennsburg, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/767,055

(22) Filed: Jun. 22, 2007

(51) Int. Cl. A61K 9/44; A61K 31/165; A61K 9/28

(52) U.S. Cl. 424/10.2; 424/474; 514/622

(57) ABSTRACT

The present invention relates to a coated dosage form having openings to expose the core material or an intermediate coating layer. The invention also relates to methods for manufacturing such coated dosage forms.

The present invention relates to a dosage form comprising a tablet core having at least two coatings that substantially surrounds the tablet core and overlap with one another at least in part. The tablet core is preferably in the form of a compressed core wherein at least one of the coatings is provided on the exterior surface of the compressed core. Openings are provided through at least one of the coatings provided over the compressed core.

### BACKGROUND OF THE INVENTION

Oral dosage forms can be provided in many forms. Solid dosage forms are commonly understood to include a range of forms from compressed powder tablets to liquid filled capsules. These dosage forms can have many shapes, colors and/or print information that serve as a means for identifying the product the dosage of active ingredient or its source. These dosage forms can also include one or more coatings that can serve, among other things, as means for product identification and/or to influence drug release characteristics.

One of the first types of film-coated elongated compressed tablets was referred to as a “caplet”. The caplet form offered enhanced swallowability over uncoated tablets due to its elongated shape and film-coated surfaces similar to that of the capsule. Gelatin coated tablets and caplets are also a well-recognized solid dosage form.

Published U.S. Patent Application 2005/0152971 relates to an improved gelatinous coated dosage form having two end regions coated with gelatinous materials and an exposed circumferential band. Openings are provided in at least the exposed band to reveal the core material. Gelatin coatings and means for providing the same are taught in a number of patents, including U.S. Pat. No. 5,234,099, which relates to a carrier apparatus for a plurality of products having a plurality of collets for maintaining the products in a fixed orientation and enable gelatin dipping.

Multi-colored dosage forms are also known as shown in U.S. Pat. No. 6,113,945 wherein a caplet or tablet core with a clear or single color uniform covering which can be applied either through an enrobing process, by spraying or by a single dip-coating step. The core itself can have a first color or be colorless, and its clear or single color covering, has the outer surface of one end or one side colored by a suitable dye to provide a two-color appearance. The dye can be applied by dipping or spray painting, with a suitable jet-spraying apparatus. In a preferred embodiment, the covering is of a clear gelatinous material. The purpose of the coloring scheme in this patent is to simulate the appearance of a gelatin dipped product.

There is a need for equipment and processes to make solid oral dosage forms having visually discernible marks or identifiers without having to utilize a printer or that can be provided with greater accuracy, consistency have greater stability have better compatibility with an array of materials, do not use the addition of solvents or excessive printing inks, or variety than presently available.

### BRIEF DESCRIPTION OF THE DRAWINGS

FIG. 1 is an enlarged isometric view of a compressed core in the form of an elongated tablet having a generally cylindrical shape, called a “gelcap core”.

FIG. 2 is an enlarged isometric view of a dosage form showing a coated tablet having a portion of the underlying coating exposed.

FIG. 3 illustrates an embodiment of the present invention.

FIG. 4 illustrates an alternative embodiment of the present invention.

### DETAILED DESCRIPTION OF INVENTION

As used herein, the term “dosage form” applies to any solid object, semi-solid, or liquid composition designed to contain a specific pre-determined amount (dose) of a certain ingredient, for example an active ingredient as defined below. Suitable dosage forms may be pharmaceutical drug delivery systems, including those for oral administration, buccal administration, rectal administration, mucosal delivery, or subcutaneous implants, or other implanted drug delivery systems; or compositions for delivering minerals, vitamins and other nutraceuticals, oral care agents, flavorants, and the like. Preferably the dosage forms of the present invention are considered to be solid, however they may contain liquid or semi-solid components. In a particularly preferred embodiment, the dosage form is an orally administered system for delivering a pharmaceutical active ingredient to the gastro-intestinal tract of a human. In another preferred embodiment, the dosage form is an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient.

As used herein the tern “tablet” refers to a solid form prepared by compaction of powders on a tablet press, as well known in the pharmaceutical arts. Tablets can be made in a variety of shapes, including round, or elongated, such as flattened ovoid or cylindrical shapes. As used herein, a “gelcap core” refers to one type of elongated, generally cylindrical or capsule-shaped tablet having straight or slightly bowed sides, and a generally circular cross-section, is and having a length to diameter ratio from about 2 to about 5, e.g. from about 2.5 to about 3.5, say about 3.

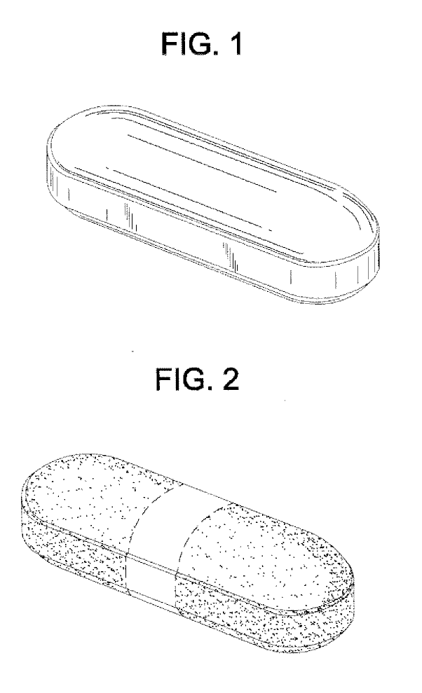

A caplet is one type of elongated tablet. There is shown in FIG. 1 a core 10 in the shape of an elongated tablet having two ends 12 at opposing sides of a longitudinal axis. A bellyband 14 occurs along the longitudinal circumference where the tablet is in contact with the die walls during compaction.

The core can have any number of pharmaceutically acceptable tablet shapes. Tablet is meant to encompass shaped compacted dosage forms in the broadest sense. An elongated tablet is a type of tablet having an elongated shape. For purposes of this application, the longitudinal axis passes through the center of both ends of the core.

The core (or substrate) may be any solid or semi-solid form. The core may prepared by any suitable method, for example the core be a compressed dosage form, or may be molded. As used herein, “substrate” refers to a surface or underlying support, upon which another substance resides or acts, and “core” refers to a material that is at least partially enveloped or surrounded by another material. For the purposes of the present invention, the terms may be used interchangeably: i.e. the term “core” may also be used to refer to a “substrate.” Preferably, the core comprises a solid, for example, the core may be a compressed or molded tablet, hard or soft capsule, suppository, or a confectionery form such as a lozenge, nougat, caramel, fondant, or flat based composition. In certain other embodiments, the core may be in the form of a semi-solid or a liquid in the finished dosage form. In embodiments in which the core is made by compression, suitable excipients include fillers, binders, disintegrants, lubricants, glidants, and the like, as known in the art. In embodiments in which the core is made by compression and additionally confers modified release of an active ingredient contained therein, such core preferably further comprises a release-modifying compressible excipient.

Suitable fillers for use in making the core by compression include water-soluble compressible carbohydrates such as sugars, which include dextrose, sucrose, maltose, and lactose, sugar-alcohols, which include mannitol, lactitol, sorbitol, imaltitol, xylitol, starch hydrolysates, which include dextrins- and maltodextrins, and the like, water insoluble plastically deforming materials such as microcrystalline cellulose or other cellulosic derivatives, water-insoluble brittle fracture materials such as dicalciuim phosphate, tricalcium phosphate and the like and mixtures thereof.

Suitable binders for making the core by compression include dry binders such as polyvinyl pyrrolidone, microcrystalline cellulose, hydroxypropylcellulose, hydroxypropylmethylcellulose, and the like; wet binders such as water-soluble polymers, including hydrocolloids such as acacia, alginates, agar, guar gum, locust bean, carrageenan, carboxymethylcellulose, tara, cum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, starches and the like; and derivatives and mixtures thereof.

Suitable disintegrants for making the core by compression, include sodium starch glycolate, cross-linked polyvinylpyrrolidone, cross-linked carboxymethylcellulose, starches, and the like.

Suitable lubricants for making the core by compression include long chain fatty acids and their salts, such as magnesium stearate and stearic acid, talc, glycerides and waxes.

Suitable clidants for making the core by compression include colloidal silicon dioxide, silicified microcrystalline cellulose such as that sold under the tradename Prosolv® by FMC Corporation and the like.

In one embodiment, the core has one or more major faces. The core may be in a variety of different shapes. For example, in one embodiment the core may be in the shape of a truncated cone. In other embodiments the core may be shaped as a polyhedron, such as a cube, pyramid, prism, or the like or may have the geometry of a space figure with some non-flat faces, such as a cone, cylinder, sphere, torus, or the like. Exemplary core shapes that may be employed include tablet shapes formed from compression tooling shapes described by “The Elizabeth Companies Tablet Design Training Manual” (Elizabeth Carbide Die Co., Inc. p.7 (McKeesport, Pa.) (incorporated herein by reference) including the following:
Shallow Concave.
Standard Concave.
Deep Concave.
Extra Deep Concave.
Modified Ball Concave.
Standard Concave Bisect.
Standard Concave Double Bisect.
Standard Concave European Bisect.
Standard Concave Partial Bisect.
Double Radius.
Bevel & Concave.
Flat Plain.
Flat-Faced-Beveled Edge (F.F.B.E.).
F.F.B.E. Bisect.
F.F.B.E. Double Bisect.
Ring.
Dimple.
Ellipse.
Oval.
Capsule.
Rectangle.
Square.
Triangle.
Hexagon.
Pentagon.
Octagon.
Diamond.
Arrowhead.
Bullet.
Shallow Concave.
Standard Concave.
Deep Concave.
Extra Deep Concave.
Modified Ball Concave.
Standard Concave Bisect.
Standard Concave Double Bisect.
Standard Concave European Bisect.
Standard Concave Partial Bisect.
Double Radius.
Bevel & Concave.
Flat Plain.
Flat-Faced-Beveled Edge (F.I.B.E.).
F.F.B.E. Bisect.
F.F.B.E. Double Bisect.
Ring.
Dimple.
Ellipse.
Oval.
Capsule.
Rectangle.
Square.
Triangle.
Hexagon.
Pentagon.
Octagon.
Diamond.
Arrowhead.
Bullet.
Barrel.
Half Moon.
Shield.
Heart.
Almond.
House/Home Plate.
Parallelogram.
Trapezoid.
Figure 8/Bar Bell.
Bow Tie.
Uneven Triangle.

Core 10 is pressed of a blend of suitable active ingredients and excipients which may be either their natural color, including, white, or can be conventionally colored as desired to provide a conventional, or elongated-shaped core of any desired color.

The dosage form of the present invention preferably contains one or more active ingredients. Suitable active ingredients broadly include, for example, pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products mucolytics, muscle relaxants, osteoporosis preparations, polydimethylsiloxanes, respiratory agents, sleep-aids, urinary tract agents and mixtures thereof.

Suitable flavorants include menthol, peppermint, mint flavors, fruit flavors, chocolate, vanilla, bubblegum flavors, coffee flavors, liqueur flavors and combinations and the like.

In another embodiment, at least one active ingredient is selected from analgesics, anti-inflammatories, and antipyretics, e.g. non-steroidal anti-inflammatory drugs (NSAIDs), including a) propionic acid derivatives, e.g. ibuprofen, naproxen, ketoprofen and the like; b) acetic acid derivatives, e.g. indomethacin, diclofenac, sulindac, tolmetin, and the like; c) fenamic acid derivatives, e.g. mefenamic acid, meclofenamic acid, flufenamic acid, and the like; d) biphenylcarbodylic acid derivatives, e.g. diflunisal, fluifenisal, and the like; e) oxicams, e.g. piroxicam, sudoxicam, isoxicam, meloxicam, and the like, f) cyclooxygenase-2 (COX-2) selective NSAIDs; and g) pharmaceutically acceptable salts of the foregoing.

In another particular embodiment of the invention, at least one active ingredient may be an analgesic selected from acetaminophen, acetyl salicylic acid, ibuprofen, naproxen, ketoprofen, flurbiprofen, diclofenac, cyclobenzaprine, meloxicam, roftecoxib, celecoxib, and pharmaceutically acceptable salts, esters, isomers, and mixtures thereof.

In another particular embodiment of the invention, at least one active ingredient may be selected from pseudoephedrine, phenylephrine, clofedianol, phenylpropanolamine, chlorpheniramine, dextromethorphan, diphenydramine, astemizole, terfeniadine, fexofenadine, loratadine, desloratadine, cetirizine, mixtures thereof and pharmaceutically acceptable salts, esters, isomers, and mixtures thereof.

In another particular embodiment, at least one active ingredient is an NSAID and/or acetaminophen, and pharmaceutically acceptable salts thereof.

The active ingredient or ingredients are present in the dosage form in a therapeutically effective amount, which is an amount that produces the desired therapeutic response upon oral administration and call be readily determined by one skilled in the art. In determining such amounts, the particular active ingredient being administered, the bioavailability characteristics of the active ingredient, the dosing regimen, the age and weight of the patient, and other factors must be considered, as known in the art. Typically, the dosage form comprises at least about 0.1 weight percent, preferably, the dosage form comprises at least about 5 weight percent, e.g. about 20 weight percent of a combination of one or more active ingredients. In one preferred embodiment, the core comprises a total of at least about 25 weight percent (based on the weight of the core) of one or more active ingredients.

The active ingredient or ingredients may be present in the dosage form in any form. For example, one or more active ingredients may be dispersed at the molecular level, e.g. melted or dissolved, amorphous or crystalline, in one or more polymorphic forms, within the dosage form, or may be in the form of particles, which in turn may be coated or uncoated. If an active ingredient is in form of particles, the particles (whether coated or uncoated) typically have an average particle size of about 1-2000 microns. In one preferred embodiment, such particles are crystals having an average particle size of about 1-300 microns. In another preferred embodiment, the particles are granules or pellets having all average particle size of about 50-2000 microns, preferably about 50-1000 microns, most preferably about 100-800 microns.

In certain embodiments, at least a portion of one or more active ingredients may be optionally coated with a release modifying coating as known in the art. This advantageously provides an additional tool for modifying the release profile of active ingredient from the dosage form. For example, the core may contain coated particles of one or more active ingredients, in which the particle coating confers a release modifying function, as is well known in the art. Examples of suitable release modifying coatings for particles are described in U.S. Pat. Nos. 4,173,626; 4,863,742; 4,980,170; 4,984,240; 5,86,497; 5,912,013; 6,270,805; and 6,322,819. Commercially available modified release coated active particles may also be employed. Accordingly, all or a portion of one or more active ingredients in the core may be coated with a release-modifying material.

In embodiments in which it is desired for at least one active ingredient to be absorbed into the systemic circulation of an animal, the active ingredient or ingredients are preferably capable of dissolution upon contact with a dissolution medium such as water, gastric fluid, intestinal fluid or the like.

In one embodiment, the dissolution characteristics of at least one active ingredient meets USP specifications for immediate release tablets containing the active ingredient. For example, for acetaminophen tablets. USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999). In embodiments in which at least one active ingredient is released immediately, the immediately released active ingredient is preferably contained in the shell or on the surface of the shell, e.g. in a further coating surrounding at least a portion of the shell. Different active ingredients have different release profiles within the USP which define “immediate release”. As used herein the term “immediate release” means the release of an active ingredient in an immediate fashion in a suitable dissolution media, i.e. more than 80% released in less than 90 minutes, e.g. less than 60 minutes.

In another embodiment, the dissolution characteristics of one or more active ingredients are modified: e.g. controlled, sustained, extended, retarded, prolonged, delayed and the like. In a preferred embodiment in which one or more active ingredients are released in a modified manner, the modified release active or actives are preferably contained in the core. As used herein, the term “modified release” means the release of an active ingredient from a dosage form or a portion thereof in other than an immediate release fashion, i.e., other than immediately upon contact of the dosage form or portion thereof with a liquid medium. As known in the art, types of modified release include delayed or controlled. Types of controlled release include prolonged, sustained, extended, retarded, and the like. Modified release profiles that incorporate a delayed release feature include pH dependent, pulsatile, repeat action, and the like. As is also known in the art, suitable mechanisms for achieving modified release of an active ingredient include diffusion, erosion, surface area control via geometry and/or impermeable or semi-permeable barriers, and other known mechanisms.

In certain preferred embodiments, the core 10 is covered with a first coating 12 and a second film-coating, 14 that can be any number of medicinally acceptable coverings. First coating 12 is provided over at least a portion of core 10. First coating 12 can be a material commonly understood by those skilled in the art as a subcoating. In one embodiment the first coating 12 or the subcoating, may optionally contain an active ingredient. The use of subcoatings is well known in the art and disclosed in, for example, U.S. Pat. No. 5,234,099, which is incorporated by reference herein. Any composition suitable for film-coating a tablet may be used as a subcoating according to the present invention. Examples of suitable subcoatings are disclosed in U.S. Pat. Nos. 4,683,256, 4,543,370, 4,643,894, 4,828,841, 4,725,441, 4,802,924, 5,630,871, and 6,274,162, which are all incorporated by reference herein. Suitable compositions for use as subcoatings include those manufactured by Colorcon, a division of Berwind Pharmaceutical Services, Inc., 415 Moyer Blvd., West Point, Pa. 19486 under the tradename “OPADRY®” (a dry concentrate comprising film forming polymer and optionally plasticizer, colorant, and other useful excipients).

Additional suitable subcoatings include one or more of the following ingredients: cellulose ethers such as hydroxypropylmethylcellulose, hydroxypropylcellulose, and hydroxyethylcellulose; polyvinyl alcohol, polycarbohydrates such as xanthan gum, starch, and maltodextrin; plasticizers including for example, glycerin, polyethylene glycol, polyvinyl alcohol; polyethylene glycol co-polymers, propylene glycol, dibutyl sebecate, triethyl citrate, vegetable oils such as castor oil, surfactants such as Polysorbate-80, sodium lauryl sulfate and dioctyl-sodium sulfosuccinate; polycarbohydrates, pigments, and opacifiers.

In one embodiment, first coating 12 comprises from about 0.1 percent to about 20, e.g. from about 1 percent to about 5 percent by weight of the core. First coating 12 is typically present in an amount, based upon the dry weight of the core, from about 0.1 percent to about 5 percent. First coating 12 can be provided by spraying in a coating pan or fluidized bed to cover the core 10 in a conventional manner. The composition for first coating 12 is optionally tinted or colored with colorants such as pigments, dyes and mixtures thereof. Pigments are generally distinguished from dyes as being insoluble in its liquid carrier, while dyes are either liquid or soluble in a selected carrier. A lake pigment is a pigment manufactured from a dye by precipitation with a metal salt. The core of a compressed dosage form are typically colored using a lake, since lakes are conducive to blending and coloring of powders.

In one embodiment, the present invention is directed to a dosage form having a core having an exterior surface, a first coating over at least part of the exterior surface of the core and a second coating over at least part of the first coating. Each of the first and second coatings contain at least one colorant and the colorant in the first and second coatings are different from one another. The colorants are alternatively opaque or translucent. At least one opening is provided through the first coating and the second coating to expose a portion of the exterior surface of the core. This dosage form can further include at least one opening that passes through the second coating and through the first coating to expose a portion of the exterior surface of the core. This dosage form can further include one or more transparent coatings that are provided over at least a portion of the second coating.

In an alternative embodiment, the present invention is directed to a dosage form having a colored core having an exterior surface, a first coating over at least part of the exterior surface of the core and a second coating over at least part of the first coating. The second coating contains at least one colorant and the colorant in the first coating is different from the color of the core. At least one opening is provided through the second coating to expose a portion of the exterior surface of the colored core. The dosage form can further include at least one opening that passes through the second coating to expose at least a portion underlying first coating. One or more transparent coatings can be provided over at least a portion of the second coating.

In an alternative embodiment, the present invention is directed to a dosage form having a core having an exterior surface, a first coating over at least part of the exterior surface of the core and a second coating over at least part of the first coating. Each of the first and second coatings contain at least one colorant and the colorant in the first and second coatings are different from one another. At least one opening is provided through the second coating to expose at least a portion of the first coating.

In an alternative embodiment, the present invention is directed to a dosage form having a core having an exterior surface, a first coating over at least part of the exterior surface of the core, and a second coating over at least part of the first coating. Each of the first and second coatings contain at least one colorant and the colorant in the first and second coatings are different from one another. At least one opening is provided through the second coating to expose at least a portion of the first coating and at least one opening is provided through the second coating to expose a portion of the exterior surface of the core. The one or more openings that pass through at least the second coating exposes less than 15%, preferably less than 10%, of the surface area of first coating and the core. A transparent third coating can be provided over at least a portion of the second coating.

In one embodiment, first coating 12 is initially applied to the entire exterior surface of core 10. First coating 12 can be applied as a clear, transparent coating such that the core call be seen. The choice is dictated by the preference of the manufacturer and the economics of the product. In a preferred embodiment, a commercially available pigment is included the subcoating composition in sufficient amounts to provide an opaque film having a visibly distinguishable color relative to the core. In certain embodiments the first coating and second coating are compositionally different.

In one embodiment core 10 is a liquid or semisolid fill of a liquid filled capsule and first coating 12 is a gelatinous coating. In this embodiment the active ingredient particles comprise about 0.1 percent to about 60, e.g. about 0.1 percent to about 20 percent by the weight of the fill. In this embodiment different capsule filling materials are used including but not limited to alkalizing agents and suitable solvents and solubilizers.

Suitable solvents and solubilizers include the chemical class of vegetable oils, vegetable oil triglycerides and triacylglycerols, specifically, for example, corn oil.

Suitable solvents and solubilizers also include the chemical class of polyglycolized glycerides, specifically, for example, lauryl macrogol 32-glycerides and steroyl macrogol 32-glycerides, such as those sold under the tradename Gelucire® 44/14 and Gelucire® 50/13 available from the Gattefosse Corporation; in addition, the chemical class of glycerol esters of fatty acids such as those sold under the tradename Gelucire® 33/01, Gelucire® 39/01, and Gelucire® 43/01 available from the Gattefosse Corporation, and mixtures thereof.

Suitable solvents and solubilizers also include the chemical class of neutral oils and triglycerides, specifically for example, medium chain triglycerides, fractionated coconut oil, caprylic and capric triglycerides such as those sold under the tradename Miglyol® 812 available from the Condea Vista Corporation, and mixtures thereof.

Suitable solvents and solubilizers also include the chemical class of polyethylene glycol and polyoxyethylene stearates, specifically, for example, polyethylene glycol 15 hydroxystearate as sold under the tradename Solutol® HS 15 available from the BASF Corporation, and mixtures thereof.

Suitable solvents and solubilizers also include the chemical class of purified vegetable, soybean and egg yolk lecithin, specifically, for example, phosphatidyl choline and 1,2-diacyl-sn-glycero-3-phosphoryl choline such as those sold under the tradename Phospholipon® 90 G available from the American Lecithin Company, and mixtures thereof.

Suitable solvents and solubilizers also include the chemical class of lecithin combined in propylene glycol, specifically, for example, standardized mixtures of phosphatidylcholile, propylene glycol, mono- and di-glycerides, ethanol, soya fatty acids and ascorbyl palmitate, such as those sold under the tradename of Phosal® 50 PG available from the American Lechitin Corporation.

Suitable solvents and solubilizers also include the chemical class of capryl-caproyl macrogol-3-glyceride and caproyl caproyl macrogol-8 glycerides such as those sold under the tradename Labrasol® available from the Gattefosse Corporation, and mixtures thereof.

Suitable solvents and solubilizers also include the chemical class of polyethoxylated hydrogenated castor oil, specifically, for example, glycerol-polyethylene glycol oxystearate, such as those sold under the tradename Cremophor® RH 40 available from the BASF Corporation, and mixtures thereof.

Suitable alkalizing agents include but are not limited to sodium bicarbonate, potassium bicarbonate, potassium hydroxide, and sodium hydroxide.

In this embodiment suitable gelatinous coatings may include film forming proteins, polymers or gums including but not limited to gelatin, iota carrageenan, lambda carrageenan, gellan gum, guar gum, xanthan gum, locust bean gum, agar, starches modified starches and mixtures thereof.

As used herein. the term “compositionally different” means having features that are readily distinguishable by qualitative or quantitative chemical analysis, physical testing, or visual observation. For example, the first and second coatings may contain different ingredients, or different levels of the same ingredients, or the first and second coatings may have different physical or chemical properties different functional properties, or be visually distinct. Examples of physical or chemical properties that may be different include hydrophylicity, hydrophobicity, hygroscopicity, elasticity, plasticity, tensile strength crystallinity, and density. Examples of functional properties which may be different include rate and/or extent of dissolution of the material itself or of an active ingredient therefrom, rate of disintegration of the material, permeability to active ingredients, permeability to water or aqueous media, and the like. Examples of visual distinctions include size, shape, topography, or other geometric features, color, hue, opacity, and gloss.

The coating may be applied to the core by any suitable method, for example by spraying, dipping, enrobing, or molding. Suitable spray-coating methods are described in, for example, U.S. Pat. Nos. 3,185,626, 4,683,256, 4,543,370, 4,643,894, 4,828,841, 4,795,441, 4,802,924, 5,630,871, and 6,274,162, the disclosures of which are all incorporated by reference herein. Suitable dipping methods are described in U.S. Pat. Nos. 4,820,524, 5,538,125; 5,228,916; 5,436,026; 5,679,406, the disclosures of which are all incorporated by reference herein. Suitable enrobing methods are described in U.S. Pat. Nos. 5,146,730 and 5,459,983. Any film former known in the art is suitable for use in the flowable material. Examples of suitable film formers include, but are not limited to, film-forming water soluble polymers, film-forming proteins, film-forming water insoluble polymers, and film-forming pH-dependent polymers. In one embodiment, the film former may be selected from cellulose acetate, ammonio methacrylate copolymer type B, shellac, hydroxypropylmetlhylcellulose, and polyethylene oxide, and combinations thereof.

Suitable film-forming water soluble polymers include water soluble vinyl polymers such as polyvinylalcohol (PVA); water soluble polycarbohydrates such as hydroxypropyl starch, hydroxyethyl starch, pullulan, methylethyl starch, carboxymethyl starch, pre-gelatinized starches, and film-forming modified starches; water swellable cellulose derivatives such as hydroxypropyl cellulose (HPC), hydroxypropylmethyl cellulose (HPMC) also known in the art as hypromellose, methyl cellulose (MC), hydroxyetlhylmethylcellulose (HEMC), hydroxyetlhylmethylcellulose (HBMC), hydroxyethylethylcellulose (HEEC), and hydroxyethylhydroxypropylmethyl cellulose (HEMPMC); water soluble copolymers such as methacrylic acid and methacylate ester copolymers, polyvinyl alcohol and polyethylene glycol copolymers, polyethylene oxide and polyvinylpyrrolidone copolymers; and derivatives and combinations thereof.

Suitable film-forming proteins may be natural or chemically modified, and include gelatin, whey protein, myofibrillar proteins, coagulatable proteins such as albumin, casein, caseinates and casein isolates, soy protein and soy protein isolates, zein; and polymers, derivatives and mixtures thereof.

Suitable film-forming water insoluble polymers, include for example ethylcellulose, polyvinyl alcohols, polyvinyl acetate, polycaprolactones, cellulose acetate and its derivatives, acrylates, methacrylates, acrylic acid copolymers; and the like and derivatives, copolymers, and combinations thereof.

Suitable film-forming pH-dependent polymers include enteric cellulose derivatives, such as for example hydroxypropyl methylcellulose phthalate, hydroxypropyl methylcellulose acetate succinate, cellulose acetate phthalate; natural resins, such as shellac and zein; enteric acetate derivatives such as for example polyvinylacetate phthalate, cellulose acetate phthalate, acetaldehyde dimethylcellulose acetate; and enteric acrylate derivatives such as for example polymethacrylate-based polymers such as poly(methacrylic acid, methyl methacrylate) 1:2, which is commercially available from Rohm Pharma GmbH under the tradenanme, EUDRAIT S, and poly(methacrylic acid, methyl methacrylate) 1:1, which is commercially available from Rohm Pharma GmbH under the tradename. EUDRAGIT L, and the like, and derivatives, salts, copolymers, and combinations thereof.

One suitable hydroxypropylmethylcellulose compound for use as a thermoplastic film-forming water soluble polymer is “HPMC 2910”, which is a cellulose ether having a degree of substitution of about 1.9 and a hydroxypropyl molar substitution of 0.23, and containing, based upon the total weight of the compound, from about 29%, to about 30% methoxyl groups and from about 7% to about 12% hydroxvlpropyl groups. HPMC 2910 is commercially available from the Dow Chemical Company under the tradename METHOCEL E. METHOCEL E5, which is one grace of HPMC-2910 suitable for use in the present invention, has a viscosity of about 4 to 6 cps (4 to 6 millipascal-seconds) at 20° C. in a 2% aqueous solution as determined by a Ubbelohde viscometer. Similarly, METHOCEL E6, which is another grade of HPMC-2910 suitable for use in the present invention, has a viscosity of about 5 to 7 cps (5 to 7 millipascal-seconds) at 20° C. in a 2% aqueous solution as determined by an Ubbelohde viscometer. METHOCEL E15, which is another grade of HPMC-2910 suitable for use in the present invention has a viscosity of about 15000 cps (15 millipascal-seconds) at 20° C. in a 2% aqueous solution as deterinined by an Ubbelohde viscometer. As used herein, “degree of substitution” means the average number of substituent groups attached to an anhydroglucose ring, and “hydroxypropyl molar substitution” means the number of moles of hydroxypropyl per mole anhydroglucose.

One suitable polyvinyl alcohol and polyethylene glycol copolymer is commercially available from BASF Corporation under the tradename KOLLICOAT IR.

As used herein, “modified starches” include starches that have been modified by crosslinking, chemically modified for improved stability or optimized performance, or physically modified for improved solubility properties or optimized performance. Examples of chemically-modified starches are well known in the art and typically include those starches that have been chemically treated to cause replacement of some of its hydroxyl groups with either ester or ether groups. Crosslinking, as used herein, may occur in modified starches when two hydroxyl groups on neighboring starch molecules are chemically linked. As used herein, “pre-gelatinized starches” or “instantized starches” refers to modified starches that have been pre-wetted, then dried to enhance their cold-water solubility. Suitable modified starches are commercially available from several suppliers such as, for example, A.E. Staley Manufacturing Company, and National Starch & Chemical Company. One suitable film forming modified starch includes the pre-gelatinized waxy maize derivative starches that are commercially available from National Starch & Chemical Company under the tradenames PURITY GUM and FILMSET, and derivatives, copolymers, and mixtures thereof. Such waxy maize starches typically contain, based upon the total weight of the starch, from about 0 percent to about 18 percent of amylose and from about 100% to about 88% of amiylopectin.

Other suitable film nforining modified starches include the hydroxypropylated starches, in which some of the hydroxyl groups of the starch have been etherified with hydroxypropyl groups, usually via treatment with propylene oxide. One example of a suitable hydroxypropyl starch that possesses film-forming properties is available from Grain Processing Company under the tradename, PURE-COTE B790In one embodiment, a suitable plasticizer may be used in the first or secondary coatings, in an amount, based upon the total dry weight of the coating, from about 0.1% to about 40%, e.g. about 1% to about 30% or from about 5% to about 20%. Examples of suitable plasticizers include, but are not limited to, polyethylene glycol; propylene glycol; glycerin; sorbitol; triethyl citrate: tributyl citrate; dibutyl sebecate; vegetable oils such as castor oil, rape oil, olive oil, and sesame oil; surfactants such as polysorbates, sodium lauryl sulfates, and dioctyl-sodium sulfosuccinates; mono acetate of glycerol; diacetate of glycerol; triacetate of glycerol; natural gums; triacetin: acetyltributyl citrate; dietbyloxalate; diethylmalate; diethyl fumarate: diethylmalonate; dioctylphthalate; dibutylsuccinate; glyceroltributyrate; glycerol monostearate; hydrogenated castor oil; substituted triglycerides and glycerides; and mixtures thereof.

The openings may be of any shape and size, and may optionally be arranged in a pattern. In embodiments in which the openings are made by laser ablation, the width or diameter of the smallest opening is typically at least 1-2 times the wavelength of light provided by the laser employed. At least a portion of the openings may be large enough to be seen with the unaided human eye, ranging in width or diameter from about 400 nanometers to as much as any dimension of the exposed subcoating. Typically, such openings will have minimum width or diameter of at least about 500 nanometers, e.g. at least about 700 nanometer, or at least about 70 microns. Typically visible openings will have a maximum width or diameter of not more than the width of the tablet, or not more than the width of the exposed subcoating band, for example not more than about 6.5 millimeters, or not more than about 3.5 millimeters, say not more than about 2.5 millimeters. Alternatively, some or all of the openings may be microscopic in size, ranking from about 1 to less than about 400 nanometers in width or diameter. In embodiments in which some or all of the openings are invisible to the unaided human eye, a plurality of openings may be arranged in a pattern that creates perforations or weak spots in the film, which facilitate disintegration. While it is not critical to the invention that the initial openings be large enough to allow the influx of water, particularly when water-soluble subcoatings are employed, it should be noted that it has been found that for certain preferred embodiments, an opening size of about 0.030 inches in width or diameter will allow water to pass therethrough. For purposes of this application, an opening is meant to refer to a generally continuous opening having a substantially uniform shape regardless of the number of layers such openings pass through. An opening “exposes” an underlying surface by making such surface visible. An opening that passes through at least one coating to a core wherein a transparent coating has been provided over the opening or an intermediate transparent layer has been provided between a top layer and the core still has “exposed” the underlying core.

In one embodiment the core, first coating 12 or the second coating 14 may contain a sensate including a flavoring agent or fragrance. Flavoring agents may include volatile flavors, non-volatile flavors, cooling agents, warming agents, low intensity sweeteners, high intensity sweeteners, salivation inducing agents or acidulants. Suitable acidulants may include acids such as citric acid, malic acid, ascorbic acid, tartaric acid, or fumaric acid. Suitable high intensity sweeteners include but are not limited to sucralose, aspartame, saccharine, acesulfame potassium and tailin. In one particular embodiment the core, first coating or secondary coatings contain different flavors, sweeteners or acidulants in order to provide a simultaneous delivery of multiple sensates.

In one embodiment the top layer coating color is dark blue, dark red, or black and the openings reveal colors from the lower layers, i.e. the core or first coating, which are lighter such as yellow or white. The contrast of these colors facilitates the identification of the dosage form.

In one embodiment, the one or more openings through the coatings do not expose greater than 15%, less than 10% of the surface area of the underlying first coat 12 and/or core 10. In one embodiment, the one or more openings through first coating 12 and second coating 14 do not expose greater than 10%, preferably less than 5% of the surthece area of underlying core 10.

In one embodiment the core is compressed and has a density of at least 0.9 g/cc, and the coatings are non-gelatinous. A s used herein “non-gelatinous” is defined as a coating which is substantially free of gelatin, i.e. less than 1.0 percent gelatin.

In one embodiment the first coating or second coating has an additional marking comprised of edible ink. The ink marking can be made using inks which are visible to the naked eye under ambient (i.e. visible light). In a separate embodiment the ink is only visible under ultraviolet light.

In one embodiment the exterior film coated surface area of first coating 12 or second coating 14 has at about 0 percent to about 20 percent, or about 0 percent to about 10 percent, e.g. about 0 percent to about 5 percent of the surface area removed via lasering.

One preferred process of manufacturing intermediate dosage form 20 begins by compressing or compacting a tablet core 10 into the desired shape of the medicament. As used herein, “compact, compacting, or compacted” and “compress, compressing, or compressed” may be used interchangeably to describe the commonly used process of compacting powders into tablets via conventional pharmaceutical tableting technology as well known in the art. One typical such process employs a rotary tablet machine, often referred to as a “press” or “compression machine”, to compact the powders into tablets between upper and lower punches in a shaped die. This process produces a core having two opposed faces, formed by contact with an upper and lower punch, and having a bellyband formed by contact with a die wall. Typically such compressed tablets will have at least one dimension of the major faces at least as long as the height of the bellyband area between the major faces. Alternately, processes have been disclosed in the prior art to enable the “longitudinal compression” of tablet cores. When longitudinally compressed tablets are employed, it has been found that an aspect ratio (height between the major faces to width or diameter of the major faces) from about 1.5 to about 3.5, e.g. about 1.9 facilitates handling.

Tablets are typically compacted to a target weight and “hardness”. Hardness is a term used in the art to describe the diametrical breaking strength as measured by conventional pharmaceutical hardness testing equipment, such as a Schleuniger Hardness Tester. In order is to compare values across differently sized tablets, the breaking strength is normalized for the area of the break (which may be approximated as tablet diameter times thickness). This normalized value, expressed in kp/cm2, is sometimes referred in the art as “tablet tensile strength.” A general discussion of tablet hardness testing is found in Leiberman et al., Pharmaceutical Dosage Froms—Tablets, Volume 2, 2nd ed., Marcel Dekker Inc., 1990, pp. 213-217, 327 329, which is incorporated by reference herein.

In certain preferred embodiments intermediate dosage form 20 produced in any of the methods described above is subsequently subjected to a mechanical or laser drilling process, A transversely excited atmosphere (TEA) laser is a preferred device for this step, particularly when used in conjunction with known tablet conveying devices, such as those commercially available from Hartnett.

In one embodiment, the coated tablets are fed into a primary hopper from which they flow via a chute into the original hopper of a “Delta” printer, available from R. W. Hartnett Company. From the original hopper, the coated tablets fall in an upright orientation, i.e. the longitudinal axis is oriented vertically into carrier links, and are conveyed upwards at about a 45-degree angle.

The coated tablets in the carrier links are conveyed between rubber impression rolls, which can be set at an “open” position. or a “printing” position. The coated tablets in the carrier links are then conveyed through a “drilling section”, in which a laser beam is rapidly pulsed, as often as every 10 microseconds, to coincide with the coated tablets passing therethrough.

The source of the laser beam is an “Impact 2015” Transverse Excited Atmosphere CO2 laser available from Lumonics Inc. The laser initially emits a 1-inch square beam having 4 Joules of energy towards a turning mirror that redirects the beam 90 degrees (upward) into a series of turning mirrors and a spherical field lens that reduces the beam from 1 inch by 1 inch to about 0.75 inch by 0.75 inch. The focused beam continues towards another turning mirror and then passes through a stainless steel mask with openings that allows only a portion of the beam to continue. The actual configuration of series the lenses and mirrors is not essential to the invention and is dictated primarily by space and cost considerations.

After passing through the mask, the patterned beam is redirected by a series of turning mirrors into a final focusing lens that reduces the size of the patterned beam about 5 times. The reduced, patterned beam ultimately strikes the coated tablets passing through the “drilling section”, causing one or more of the coatings to be ablated and form shaped openings in a pattern determined by the mask. Adjusting the height of the final turning mirror can modify the striking position of the patterned beam. Mirrors and lenses are commercially available from companies, such as LightMachinery, Inc.

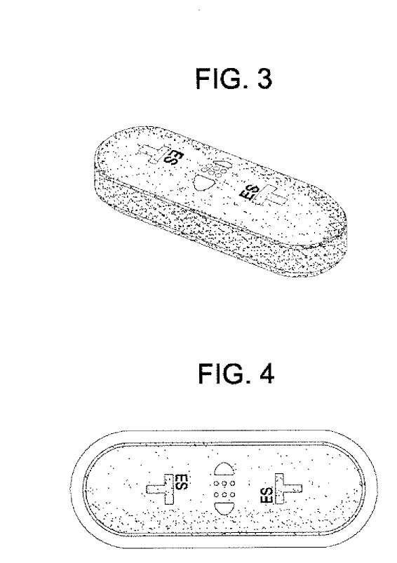

FIG. 3 illustrates final dosage form 30 having at least two coatings 24 (first coating) and 26 (second coating). In one embodiment, openings 32 are provided through second coating 26 and first coating 24 that exposes an overcoated exterior surface of core 10. In another embodiment, openings 32 are provided through second coating 26 and first coating 24 to expose a portion of an overcoated exterior surface of core 10 and further openings 33 are provided through only second coating 26 to expose a portion of first coating 26. In yet another embodiment, openings 32 are provided through second coating 26 and first coating 24 to expose a portion of an overcoated exterior surface of core 10 and further openings 33 are provided through only second coating 26 to expose a portion of core 10.

One or more openings 32/33 are provided using a mechanical drill or laser. In another embodiment, the mechanical drill or laser produces at least one, preferably a plurality of openings 32/33 through first coating 24, first coating 24 and second coating 26, or combinations thereof. In certain optional embodiments, openings 32/33 are large enough to be visible to the naked human eye. In this case, those skilled in the art can appreciate the advantage of using first coating 24 and/or second coating 26 and/or core 10 having a color(s) that are different in order to highlight the presence of openings 32/33.

The color difference can result from inclusion of a colorant or coloring agent in first coating 24, second coating 26 and/or core 10. In an alternative embodiment, the colorant or coloring agent is incorporated into compacted material used to make core 10, while first coating 24 and/or second coating 26 have one or more different colors from core 10.

The coloring agent can be added in the form of a water soluble dye, or a lake and with or without the use of an opacifier. Suitable opacifiers include but are not limited to titanium dioxide or mica.

Any variety of markings may be made in a variety of embodiments. In one embodiment the laser making is used to provide readable numerical or written characters such as defined by ASCII (American Standard Character Information Interchange) DEC code values #49 to #57 (numbers); #65-78 and #80-90 (upper case letters); and #97-110 and #112 to #122 (lower case letters), to communicate active ingredient types, product identity, company names (or abbreviations), lot or batch numbers, dates or dosage amounts without limitation to font type, size or presentation format. ASCII (American Standard Code for Information Interchange) is a character encoding based on the English alphabet. ASCII codes represent text in computers, communications equipment, and other devices that work with text. Most modern character encodings which support many more characters have a historical basis in ASCII.

In one embodiment the laser mark provides a textural difference that can be perceived through touch by the finger or tongue. This may be especially advantageous to those who may read characters through the use of Braille. Protrusions and indentations can traditionally be produced in dosage forms through tooling, which is imprinted to a tablet during compression. In the laser marking embodiments, the mark is provided in a more precise fashion, e.g., wherein the diameter of the character is smaller than 5 mm, or smaller than 1 mm, e.g. smaller than 0.5 mm. In this embodiment the depth is greater than 0.05 mm e.g. greater than 0.1 mm. In another embodiment the laser mark provided is present in the form of a barcode. In another embodiment the laser mark is used to provide a character, picture or a mixture of a picture, character and product information.

In certain types of ink printing applications on pharmaceutical dosage forms, it is difficult to produce a substantially legible marking using light inks on dark tablets. In such eases the light inks tend to blur or run causing illegible makings.

In one embodiment of this invention the Munsell color system value of lightness can be used to differentiate between the color of the core and the color of the first or second coatings. The Munsell color system is used in colorimetry to define color space that specifies colors based on three color dimensions; hue, value (or lightness), and chroma (roughly saturation). In this embodiment the color of the core is lighter than the color of the coating so that the definition of the marking can be clearly read when lasered into the surface of the coating and upon exposure of the core. In order to further define this embodiment, the core color has a lightness value of 8.5 to 10 on the Munsell scale, and the coating color has a darkness of zero (0) to 2.5. In this embodiment the marking must be legible such that a person of at least 20/20 vision or better can recognize and read the ASCII image(s) formed by absence of coating(s) with the exposure of the core at a distance from the surface of the dosage form to the observer of about 18 inches. In a more specific embodiment the ASCII image(s) which are visible to an observer at about 18 inches are from about 1 to about 10 mm in length, e.g. about 1 mm to about 6 mm in length, e.g. about 2 mm to about 4 mm in length.

It will become apparent to those skilled in the art that various modifications to the preferred embodiments of the invention can be made by those skilled in the art without departing from the spirit or scope of the invention as defined by the appended claims.

The present invention is further illustrated by the following non-limiting example.

### EXAMPLE 1

### Preparation of White Placebo Caplet and Round Tablet Blend

Manually pass 18,562.5 grams of microcrystalline cellulose and carboxymethylcellulose sodium NF commercially available from the FMC corporation as Avicel pH 102® and 6250 grams of pregelatinized starch commercially available from Colorcon corporation as Starch 1500® through a 20 mesh screen and combine in a suitable plastic bag. Combine 187.5 g of magnesium stearate with approximately one third of the Avicel/Starch mixture and pass through a 20 mesh screen. Add half of the remaining Avicel/Starch mixture to a 2 quart V-blender followed by the magnesium stearate/Avicel/Starch mixture and the remaining Avicel/Starch mixture and blend for 5 minutes.

Part A: Use approximately half of the above blend to compress white placebo tablets on a rotary tablet press equipped with 27/64″×0.081″ round tooling. Compress the tablets at a weight of approximately 492 mg, a thickness of about 7.12 mm and a hardness of about 9.3 kp.

Part B: Use approximately half of the above blend to compress white placebo caplets on a rotary tablet press equipped with 687.5″×281.2″ simulated capsule shaped (“caplet”) tooling. Compress the caplets at a weight of approximately 502 mg, a thickness of about 7.30 mm, and a hardness of about 8.5 kp.

### EXAMPLE 2

### Preparation of Orange Placebo Caplet and Round Tablet Blend

Manually pass 18,437.5 grams of microcrystalline cellulose and carboxymethylcellulose sodium NF commercially available from the FMC corporation as Avicel pH 102®, 6250 grams of pregelatinized starch commercially available from Colorcon corporation as Starch 1500®, and 125.0 grams of FD&C Yellow #6 through a 20 mesh screen and combine in a suitable plastic bag. Combine 187.5 g of magnesium stearate with approximately one third of the Avicel/Starch/Yellow #6 mixture and pass through a 20 mesh screen. Add half of the remaining Avicel/Starch/Yellow #6 mixture to a 2 quart V-blender followed by the magnesium stearate/Avicel/Yellow #6/Starch mixture and the remaining Avicel/Starch/Yellow #6 mixture and blend for 5 minutes.

Part A: Use approximately half of the above blend to compress orange placebo tablets on a rotary tablet press equipped with 27/64″×0.081″ round tooling. Compress the tablets at a weight of approximately 484 mg, a thickness of about 7.05 mm, and a hardness of about 10.2 kp.

Part B: Use approximately half of the above blend to compress orange placebo caplets on a rotary tablet press equipped with 687.5″×281.2″ simulated capsule shaped (“caplet”) tooling. Compress the caplets at a weight of approximately 506 mg, a thickness of about 7.27 mm, and a hardness of about 8.6 kp.

### EXAMPLE 3

### Preparation of Black Film Coating Solution

Add 1760 g of sterile water for irrigation to a 5 liter stainless steel vessel. Set a Lightning laboratory mixer to 50 RPM and add 440.0 grams of hypromellose based film coating polymer containing black colorant, commercially available from the Colorcon corporation as Opadry® mix for 45 minutes.

### EXAMPLE 4

### Black Film Coating of Cores

Add 2.75 kg of tablets and caplets from each of Example 1, Part A (white tablets) and Part B (white caplets), and Example 2, Part A (orange tablets) and Part B (orange caplets); for a total of 11 kg to a 24 inch vented (Acela Cota) coating pan. Spray coat the batch at a spray rate of approximately 44 grams per minute, about 14 RPM, an inlet air temperature of about 85° C. and an atomization air pressure of about 55 psi. Spray 1500 grams of the coating solution, which is equivalent to 300 g of dried coating, or about a 2.7% weight gain.

### EXAMPLE 5

### Preparation of Silver Coating Solution

Add 185 g of sterile water for irrigation to a 1 liter stainless steel vessel. Set a lightning laboratory mixer to 50 RPM, add 15.0 grams of hypromellose based film coating polymer containing silver colorant, commercially available from the Colorcon corporation as Opadry® and mix for 45 minutes.

### EXAMPLE 6

### Silver Top Coating of Cores

Add 1 kg of black coated tablets and caplets from Example 4 (250 g of each) to a 15″ Compu-Lab tablet coating unit and spray coat at a spray rate of about 7 g/minute, an inlet air temperature of about 75° C., an atomization air pressure of 20 psi, and 15 RPM for an equivalent of 1.5% weight gain.

### EXAMPLE 7

### Red-Yellow Top Coating of Cores

Repeat the experiment in Example 6 with a red-yellow top coating solution, wherein the coating is a hypromellose based coating commercially available from the Colorcon corporation as Opadry® containing the colorant Yellow #6 for an equivalent of 1.5% weight gain.

### EXAMPLE 8

### Laser Drilling of Tablets

Drill tablets from Examples 4, 5, 6 and 7 with holes to expose layers underneath. Use a Transverse-Excited Atmospheric (TEA) CO2 laser to drilling through the film coatings. Use a wavelength of approximately 10.6 nanometers, and a pulse duration of approximately 10 microseconds. Any shape hole can be produced by means of placing a mask in the path of the laser beam. For the sake of ease of calculations, a simple circle is used. For purposes of an Example, the name “Motrin 100 mg” is drilled. The diameter size of the hole on the tablet can be varied from 1.5 mm to 2.0 mm. The larger the area ablated by the laser, the more energy required.

Drill two sets of holes through the Tablets from Example 6. In the first set of holes, drill to the core to reveal the orange color in the core. In the second set of holes, drill only through the first layer to revealing the black first coating layer. A laser intensity of approximately 0.15-0.20 Joules/mm² achieves total ablation of the outer coating (exposing the inner coating) in one pulse. A laser intensity of approximately 0.4-0.8 Joules/mm² achieves total ablation of the outer and inner coating (through to expose the core) in one pulse.

### EXAMPLE 9

### Laser Drilled Coated Tablets with Printed Identification

The Black Film Coated laser drilled caplet placebo dosage form from Example 4 (Single Layer coated) and Example 8 (subsequently laser drilled with the words “Motrin 100 mg”) are marked with additional printing by passing through a Hartnett Delta tablet printer, and printed using, silver edible ink with the words “Store at RT”, wherein RT indicates Room Temperature.

I/we claim:

1. A dosage form comprising:
a) a core having an exterior surface:
b) a first coating over at least part of the exterior surface of the core; and
c) a second coating over at least part of the first coating:
wherein each of the first and second coatings contain at least one colorant and the colorant in the first and second coatings are different from one another; and wherein at least one opening is provided through the first coating and the second coating to expose a portion of the exterior surface of the core.

2. A dosage form according to claim 1 further comprising at least one opening through the second coating and through the first coating to expose a portion of the exterior surface of the core.

3. A dosage form according to claim 1 further comprising a transparent third coating that is provided over at least a portion of the second coating.

4. A dosage form according to claim 1, wherein the core is a compressed tablet.

5. A dosage form according to claim 1, wherein the compressed tablet has an elongated shape.

6. A dosage form according to claim 1, wherein printed matter is provided on the exterior surface of at least one of the first and second coatings.

7. The dosage form of claim 1, wherein the core comprises acetaminophen and from which at least about 90% of the acetaminophen is released after 6 minutes in USP pH 5.8 phosphate buffer in USP Apparatus II (paddle method) at 50 rpm.

8. A dosage form comprising:
a) a colored core having an exterior surface;
b) a first coating over at least part of the exterior surface of the core; and
c) a second coating over at least part of the first coating;
wherein the second coating contains at least one colorant and the colorant in the first coating is different from the color of the core; and
wherein at least one opening is provided through the second coating to expose a portion of the exterior surface of the colored core.

9. A dosage form according to claim 8, wherein at least one opening passes through the second coating to expose at least a portion underlying first coating.

10. A dosage form according to claim 8, wherein printed matter is provided on the exterior surface of at least one of the first and second coatings.

11. A dosage form according to claim 8 further comprising a transparent third coating that is provided over at least a portion of the second coating.

12. The dosage form of claim 8, wherein the core comprises acetaminophen and from which at least about 90% of the acetaminophen is released after 6 minutes in USP pH 5.8 phosphate buffer in USP Apparatus II (paddle method) at 50 rpm.

13. A dosage form comprising:
a) a core having an exterior surface;
b) a first coating over at least part of the exterior surface of the core; and
c) a second coating over at least part of the first coating;
wherein each of the first and second coatings contain at least one colorant and the colorant in the first and second coatings are different from one another; and
wherein at least one opening is provided through the second coating to expose at least a portion of the first coating.

14. The dosage form of claim 13, wherein the core comprises acetaminophen and from which at least about 90% of the acetaminophen is released after 6 minutes in USP pH 5.8 phosphate buffer in USP Apparatus II (paddle method) at 50 rpm.

15. A dosage form comprising:
a) a core having an exterior surface;
b) a first coating over at least part of the exterior surface of the core; and
c) a second coating over at least part of the first coating;
wherein each of the first and second coatings contain at least one colorant and the colorant in the first and second coatings are different from one another; and
wherein at least one opening is provided through the second coating to expose at least a portion of the first coating and at least one opening is provided through the second coating to expose a portion of the exterior surface of the core; and wherein the one or more openings through at least the second coating exposes less than 15% of the surface area of first coating and the core.

16. A dosage form according to claim 15 further comprising a transparent third coating that is provided over at least a portion of the second coating.

17. A dosage form according to claim 15, wherein printed matter is provided on the exterior surface of at least one of the first and second coatings.

18. A dosage form according to claim 15, wherein the first or second coating is non-gelatinous.

19. The dosage form of claim 15, wherein the core comprises acetaminophen and from which at least about 90% of the acetaminophen is released after 6 minutes in USP pH 5.8 phosphate buffer in USP Apparatus II (paddle method) at 50 rpm.

20. A dosage form according to claim 15 further comprising at least one opening through the second coating and the first coating to expose a portion of the exterior surface of the core.

21. A dosage form comprising:
a) a colored liquid or semi-solid core having an exterior surface;
h) a first gelatinous coating over at least part of the exterior surface of the core: and
c) a second coating over at least part of the first coating;
wherein the second coating contains at least one colorant and the colorant in the second coating is different from the color of the first coated core; and
wherein at least one opening is provided through the second coating to expose a portion of the exterior surface of the coated colored liquid or semi-solid core.

22. A method for producing a dosage form comprising:
a) providing a first coating having a first color over at least a part of an exterior surface of a solid core;
b) providing a second coating having a second color over at least a part of the first coating;
c) providing at least one opening through the second coating to expose a portion of the first coating.

23. A method according to claim 22, wherein the at least one opening through the second coating is provided by removing a portion of the second coating using a laser.

24. A method for producing a dosage form comprising:
a) providing a first coating over at least a part of an exterior surface of a solid core having a first color;
b) providing a second coating having a second color over at least a part of the first coating;
c) providing at least one opening through the second coating to expose a portion of the solid core.

25. A method according to claim 24, wherein the at least one opening through the second coating is provided by removing a portion of the second coating using a laser.

26. A method for producing a dosage form comprising:
a) providing a first coating having a first color over at least a part of an exterior surface of a core;
b) providing a second coating having a second color over at least a part of the first coating; and in no particular order,
c) providing at least one opening through the second coating to expose a portion of the first coating;
d) providing at least one opening through at least the second coating to expose a portion of the core.

27. A method according to claim 26, wherein the at least one opening through the second coating to expose a portion of the first coating is provided by removing a portion of the second coating, using a laser that is operating at a first energy level and the at least one opening through at least the second coating to expose a portion of the core is provided by removing a portion of the second coating using a laser that is operating at a second energy level.

28. A method according to claim 26, wherein the at least one opening through the second coating to expose a portion of the first coating is provided by removing a portion of the second coating using a laser having a first filter that produces a beam having a first energy level and the at least one opening through at least the second coating to expose a portion of the core is provided by removing a portion of the second coating using a laser at an energy level different from the beam exiting from the first filter.

29. A method of identifying dosage forms with unique characters and colors wherein the dosage form has a) a core having an exterior surface:
a) a first coating over at least part of the exterior surface of the core; and
b) a second coating over at least part of the first coating;
wherein each of the first and second coatings contain at least one colorant and the colorant in the first and second coatings are different from one another; and
wherein at least one opening is provided through the second coating to expose a portion of the exterior surface of the core.

30. A dosage form comprising:
a) a core having an exterior surface;
b) a single film coating over at least 80% of the exterior surface of the core; and
c) an image formed by the absence of the film coated layer exposing the core which is composed of at least two or more characters as defined by a subset of ASCII characters with DEC code values of #49 to #57 (numbers): #65-78 and #80-90 (upper case letters); and #97-110 and #112 to #122 (lower case letters).

31. A dosage form according to claim 30, wherein the image is visually detected by detecting a difference in color.

32. A dosage form according to claim 30, wherein the core color is non-white.

33. A dosage form according to claim 30, wherein the active ingredient is immediate release.

34. A method for making the dosage form according to claim 30, wherein a laser is used to remove up to 20% of the exterior surface resulting in an image formed by absence of film coating.

35. A method for making the dosage form according to claim 30 wherein a laser is used to remove up to 20% of the exterior surface resulting in an image formed by absence of film coating.

37. A dosage form according to claim 30, wherein the dosage form has an additional marking on the film coated portion comprised of ink.

38. A dosage form according to claim 30, wherein the core has a lightness value of 8.5 to 10 and the coating has a lightness value of zero to 2.5.

39. A dosage form according to claim 34, wherein the ink cannot be detected by the naked eye but is visible only under ultraviolet light.

40. A dosage form according to claim 31, wherein an additional marking on the film coated portion formed by a layer of ink can be detected by the human eye.
